(19) United States
(12) Patent Application Publication
(10) Pub. No.: US 2026/0106006 A1
(43) Pub. Date: Apr. 16, 2026

(54) System & Method for Supporting Speech Language Pathology Practices

(71) Applicant: Speak2me Inc., Los Angeles, CA (US)

(72) Inventors: George Ehrlich, Santa Monica, CA (US); Lucas T. Warner, Lancaster, CA (US); Rotem Avisar, Beachwood, OH (US)

(73) Assignee: Speak2me Inc., Los Angeles, CA (US)

(21) Appl. No.: 18/913,986

(22) Filed: Oct. 11, 2024

Related U.S. Application Data
(60) Provisional application No. 63/543,680, filed on Jan. 1, 0001.

(51) Int. Cl. G16H 20/00; G06V 40/16; G09B 19/04; G16H 15/00; G16H 40/67; G16H 50/20

(52) U.S. Cl. CPC G16H 20/00; G06V 40/174; G09B 19/04; G16H 15/00; G16H 40/67; G16H 50/20

(57) ABSTRACT

A system and method for supporting speech language pathology practices that utilizes a patient smart device for recording patient exercises at an arbitrary location of the patient for generating patient exercise data. The patient exercise data with accompanying meta data is transmitted to a central server for processing and storage. The processing, which can include the use of artificial intelligence and learning applications, generates one or more assessments of patient progress and other status information that is transmitted to a smart device of a medical provider for review by the medical provider and for updating the patient therapy, if necessary.

### CROSS-REFERENCES TO RELATED APPLICATIONS

This application claims the benefit of U.S. provisional patent application Ser. No. 63/543,680 filed on Oct. 11, 2023 (the “'680 application”), incorporated herein by reference.

The disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

There are various causes of speech pathologies, including brain damage from strokes or other interference with blood flow from the brain. In such circumstances, the patient needs therapy to rehabilitate the patient's speech capabilities.

The medical profession has developed specific treatments for rehabilitating patient speech functions. Typically, this requires the patient to visit a speech therapist (or similar type of professional) at a clinic for diagnosis, monitoring, and treatment. The therapist will provide the patient with exercises and other practices and therapies, for use outside the clinic (e.g., “homework”), that are designed to improve the patient's speech functionality with regular practice. Regular visits to the therapist at the clinic are traditionally required in order to monitor patient progress and to determine whether the therapy is working properly. Such regular visits add to the medical expenses of the patient.

However, it is not unusual for a patent to have problems properly implementing the exercises, or the lack of progress may lead to a need for additional or other types of exercises to be performed. But under current practice, the therapist can only determine these problems by having the patient visit the therapist at the clinic (or alternatively having the therapist visit the patient at the patient's home), where the therapist can examine the patient's speech performance by observation, and, by remembering prior examinations, determine if any progress is being made. This is a time consuming practice that puts burdens on both the therapist and the patient, and often leads to delays in identifying problems with the current therapy. Also, relying on the therapist's memory of prior examinations may lead to errors or memory issues. Improvements to solve these, and other, problems, and make the therapy more effective and more efficient, are desirable.

### SUMMARY

Provided are a plurality of example embodiments, including, but not limited to, a system configured to aid in speech therapy by providing the patient with tools to record and transmit images and audio of various exercises and practices performed by the patient in the patient's own home. The images are analyzed using a smart system, and the results and original images along with suggestions and progress reports can be provided to the patient's speech therapist to aid in the treatment of the patient.

Also provided is a method of evaluating a speech therapy treatment for supporting the speech therapy of a patient, comprising the steps of:

Executing software on a patient smart device for performing the steps of: providing one or more speech exercises to the patient; the patient performing said one or more speech exercises using the patient smart device at an arbitrary location of the patient; said patient smart device monitoring the performance of the patient performing said one or more speech exercises, said monitoring including capturing responses of the patient while performing the exercises to generate patient exercise data; and the smart device sending the patient exercise data over a communication network to a remote server;

Said remote server receiving said patient exercise data;

Said remote server executing software for performing the steps of: analyzing said patient exercise data for determining patient progress; generating assessment data of the patient progress based on said analyzing; and the server sending the assessment data over a communication network to a medical provider smart device of a medical provider located remotely from the patient;

And said medical provider smart device providing said assessment data for access by the medical provider to review the progress of the patient regarding the speech therapy treatment.

Also provided are additional example embodiments, some, but not all of which, are described hereinbelow in more detail.

### BRIEF DESCRIPTION OF THE DRAWINGS

The patent or application file contains at least one drawing executed in color. Copies of this patent or patent application publication with color drawings will be provided by the Office upon request and payment of the necessary fee.

The features and advantages of the example embodiments described herein will become apparent to those skilled in the art to which this disclosure relates upon reading the following description, with reference to the accompanying drawings, in which:

FIG. 1 shows an example top-level diagram of a system for implementing the invention.

FIG. 2 shows an example distribution of the functionality of an example system.

FIG. 3 is a set of photographs showing example Oral Motor Exercises.

FIG. 4 is a set of example prompted sentences.

FIG. 5 is a graphic showing an example Asymmetrical front-facing Idle Position.

FIG. 6 shows an example symmetrical front-facing idle position.

FIG. 7 shows an example idle position of the left sphere.

FIG. 8 shows an example idle position of the right sphere.

FIG. 9 shows an example Asymmetrical Idle Position of the Left Sphere.

FIG. 10 shows an example Asymmetrical Idle Position of the Right Sphere.

FIG. 11 shows an example face mesh.

FIG. 12 shows a table of the monitored data points and their movement in the mesh of FIG. 11.

FIGS. 13A-13C show example facial masks of an example patient over a period of time of one year.

FIG. 13D shows an example alternative position of the facial masks of FIGS. 13A to 13C.

FIGS. 14A-14C show example facial masks of an example patient with color processing of an example patient over a period of time of one year.

FIG. 15 is an example flow chart of an example method of supporting speech therapy.

FIG. 16 is block diagram of an example architecture for an example embodiment for practicing a speech therapy support method.

Appendix A, incorporated herein by reference, provides additional figures and photos showing example features of example embodiments.

### DETAILED DESCRIPTION OF THE EXAMPLE EMBODIMENTS

Patients who have problems with speech caused by strokes or other pathologies often undergo procedures to improve their speech problems by vising a therapist or other professional, who develops a treatment plan to improve the speech capabilities of the patient. For example, In the case of stroke patients, many doctors and therapists have no measurable way to determine a patient's facial droop. Instead they have to provide an estimate based on their best guess of how one side of the face sags. This discussion is solely based on the clinicians experience, personal judgment and familiarity with facial symmetry. This method is highly subjective, inconsistent and has lots of margin for error.

Under traditional practice, the therapist reviews the patient's medical status, and will first analyze the patient for problems that may occur in the patient's speech capabilities, including stuttering; an inability to recite words; and forgetting words, for example, by having the patient attempt to talk to the therapist (which man involve questioning by the therapist).

Based on the subjectivity of facial droop estimation, there is a need for a quantitative solution. By using 3D facial scans provided by the disclosed technique using, in one embodiment, Apple's ARKit's 10,000 Point facial mesh, the technique is able to measure the degree of facial droop directly and over time. The process is then able to generate heat maps that illustrate the range of motion in each face. Heat maps can also be used to illustrate the healing process of facial droop over a period of time. This will ensure that:
Reduced reliance upon deductions and estimations.
Less Human error.
Clinicians can share the same dataset for more consistent patient treatment.
Precise and consistent tracking of progress over time from even the most finite detail.

For each new patient, the inventive process provides an interface with the patient to map their face and take a baseline scan. Such a scan can be initialized from the mobile application executing a smart device of the patient. The baseline is basically the first scan a patient takes to set the starting point. Over time, a comparison can check the patient's first scan (Baseline) against all the subsequent scans following the baseline scan to monitor a patient's progress. Over time, as the patient improves, the patient's face will slowly recover from the droop and they will get more ranges of motion in the affected area. This improvement will be noticeable even at the slightest level in all the subsequent scans.

Another comparison can be done between a machine learning trained face. A face that takes the average of hundreds or thousands of healthy faces (without facial droops) to obtain a standard face for comparison purposes. Comparing the patient's facial droop against this generated standard model helps determine (and measure) the facial droop of the patient and simulate what recovery COULD look like in ideal situations.

The application will instruct the patient how to position their face as well as turning their face in 4 directions (up, down, left, right), as well as walking them through a few facial deformations (e.g. Smile, Open Mouth, Grit Teeth). Using the devices front camera and, for example, ARKit's facial recognition technology, the scan data is collected to generate a profile based off of the collected information. The scan will map the patient's features with specific focus on the cheek and eye areas. This can be used to compare future scans of the patient's face for comparing results, such as determining any improvements in the patient. For HIPAA compliance, this profile can be designed to not be tagged with any of the patient's personal information but instead a specific numeric ID.

At regular intervals, and/or when the patient visits the treating medical facility, the patient can have their profile updated with their latest facial data. The process to do this can be identical with generating the Baseline Profile. This will be saved within the same profile with metadata, such as the date and time. Operators will be able to use the on-screen controls inside of an application on a viewing computing device to morph the scanned face mask to any desired date that a scan was taken and visualize the changes directly on the face mask in real time as well as generate a heatmap (with color coded information) that better illustrates the range of motion. In this way, the patient's progress during treatment can be monitored.

The operation of the patient's facial muscles and expressions are examined, tested, and monitored using video and exercises. Appendix A, of the '680 application, incorporated by reference and filed herewith, shows various approaches that can be used to provide speech therapy to patients that can be enhanced by the inventive methods and devices disclosed herein. These include:

Providing an aphasia treatment utilizing: Picture Cards; Worksheets; Caregiver Education with home programming; Group therapy to work on social skills; utilizing computer applications (including those provided by the invention that many include supporting these other tools); Intonation Therapy (e.g., singing); Script Training; Oral-Motor Exercises; Word Finding approaches; etc.

As part of these treatments, the therapist can utilize various activities to determine patient progress, including:

Picture Description activities: where Patients are shown a flashcard image (e.g., 3×5 cards as shown in page 2 of Appendix A) and they are directed to describe what they see. The disclosed system can be adapted to display flashcards to the users on a user device, and record their descriptions.

Oral Reading for Language in Aphasia (ORLA), which is a treatment for individuals with aphasia that involves repeated practice reading sentences aloud with the clinician to improve reading comprehension via phonological (sounds heard) and semantic (synonym) reading routes. Appendix A shows example sentences on page 3. This treatment focuses on prosody, speech, and reading skills. This doesn't typically need training for this task. The disclosed system can be adapted to display sentences for reading and then record the patient's responses. The system can utilize Phonological Components Analysis (PCA) by utilizing worksheets, for example.

Verb Network Strengthening Treatment (VNeST): This approach targets verbs and their roles to activate semantic networks and to improve the production of basic syntactic structures (e.g., subject-verb-object). For example, the patient is given a verb (e.g., measure) and is asked to answer “who” ______ “what”? (e.g., a “carpenter measure wood”). This is often done on notecards, a whiteboard, or worksheets, or via teletherapy. The disclosed system can be adapted to display or voice verbs to the patient and record the patient's responses.

Phonological Components Analysis (PCA): This word-finding treatment that helps the patient learn to analyze the sounds in word to aid in word retrieval. For example, worksheets given as homework, such as the example shown on page 5 of Appendix A. The disclosed system can be adapted to provide the homework worksheets for the patient to fill out via typing or recording voice responses.

Home programming: Identify nouns vs verbs. Deciding which words in the sentence is the noun vs the verbs: “The woman is smelling the flowers”; “They are planting the flowers”; “The boy is watering the flowers”. The disclosed system can be adapted to display or recite sentences and record patient responses identifying the nouns and verbs.

The disclosed system can be adapted to record oral motor exercises provided by the therapist, and send regular updates to the therapist including both videos of the exercises and expert analysis (e.g., utilizing Artificial Intelligence) of the results. Pages 7 and 8 of Appendix A shows an example of the visual images provided by such oral exercises. Page 9 of Appendix A show and describe the muscles involved in facial expressions and communications that are utilized in these exercises. By recording patient's faces during these exercises being performed remotely (e.g., at home), the system of the invention can record and analyze the patient's progress for providing to the therapist for review and updating treatments and patient training, where appropriate. The system can utilize AI for suggesting such changes in therapy and implement machine based learning.

The system can support Language Based Therapy (e.g., Tactus Therapy) that includes the following: (1) Naming Therapy: Think of the word; Describe the word's attributes; Say the name; Improve word retrieval; Ability to personalize; (2) Reading Therapy: Reading silently or aloud; Reading phrases & sentences; Making Choices; Attention to Detail; and (3) Writing Therapy: e.g. Spelling Skills and writing skills.

Tactus offers Apraxia Therapy for severe aphasia utilizing the inventive system, as shown on page 11 of Appendix A for analyzing motor movements. The patient is directed to say a common sequence (I.e., counting to ten, reciting the alphabet, etc.) and a pre-recorded image of a woman's mouth is used to model target phrase/sequence. (1) The patient first listens to the prompt, (2) The patient attempts to say the phrase with the audio and visual demonstration, (3) The patient then says the target phrase again without audio (still seeing visual); and (4) finally a recording of the verbal outfit is recorded and played back for the patient. The patient can be requested to rate their output (e.g., selecting a frowny face, okay face, smiley face) The disclosed system can be adapted to support all of these steps, and also record the results for analysis and providing to the therapist.

Patient Improvements discussed in Appendix A can include: Interactive and engaging activities; Immediate feedback on all activities-most importantly voice/motor movement; Effective evidence-based activities; Ability to contact provider (SLP) for questions and/or additional support; Activities with specific directions that can be done anytime and anywhere; Patient will be able to see their face during oral-motor exercises to review to allow patients to see improvements; Immediate feedback on vocal prosody, fluency, and intonation; Gamified activities are highly motivating to increase user return rate and impact.

Improvements for the Speech Pathologist (therapist) include: Effective evidence-based activities; Ability to reach more patients; The Technology records data on motor movements, patterns, and any asymmetry that is not evident by the naked eye; Heat mapping technology to pinpoint energy/muscle tension within the face during activities; Heat mapping technology mirrors the color scheme used in PET scan for easy clinician understanding; Facial mapping allows data to be collected on asymmetries to create a baseline for patients to see progress over time; Collected data will also be provided to clinician as quantitative baseline data compared to subjected data used today; Quantitative data is collected to evaluate improvements over time; The Technologies offer SLP's quantitative data on patient's performance at a glance, alleviating the clinician's need to compute scores into percentages and creating more objective data for growth comparisons.

Note that for any of the above exercises, the system can record the patient's responses by receiving typed text, recording patient vocal descriptions, and/or recording the patient's video during the response. The latter approach is particularly useful because it can provide the therapist with visual evidence of the patient's progress and status of muscle usage.

The inventive system is particularly useful because patient improvement relies on repetition, but the system can monitor the patient to ensure that the patient is properly performing the exercises and showing improvement. In addition, the system better ensure success because it provides the patient: Interactive and engaging activities; Immediate feedback on all activities-most importantly voice/motor movement; Effective evidence-based activities; Ability to contact provider {SLP) for questions and/or additional support; Activities with specific directions that can be done anytime and anywhere; The patient will be able to see their own face during oral-motor exercises to review to allow patients to see improvements; Access to interactive, engaging, and effective speech therapy tasks; Provide immediate feedback on vocal prosody, fluency, and intonation; and Provide gamified activities are highly motivating to increase user return rate and impact.

In addition, the system provides the therapist (e.g., speech pathologist) with: Effective evidence-based activities; Objective data collection inputted into the (Electronic Medical Record) EMR system; Ability to reach more patients and an Increase productivity rate, i.e., streamline patient throughput; Billable (remote) teletherapy options; Recordings of data on motor movements, patterns, and any asymmetry that is not evident by the naked eye; Decrease copious amounts of storage by streamlining home programming (homework); Heat mapping technology to pinpoint energy/muscle tension within the face during activities; Innovative Heat Mask Technology; Heat mapping technology mirrors the color scheme used in PET scan for easy clinician understanding; Facial mapping allows data to be collected on asymmetries to create a baseline for patients to see progress over time; Collected data will also be provided to clinician as quantitative baseline data compared to subjective data used currently; Quantitative data is collected to evaluate improvements over time; Capitalize on peak brain plasticity time to yield in the best results (support principles of brain plasticity including repetition and intensity); Eliminates the need to travel to appointments; Provide a direct link between patients and their SLPs; Provide access to interactive, engaging, and effective speech therapy tasks; Provide immediate feedback and objective progress tracking and analysis including vocal output; and The improved technologies offers SLP's quantitative data on patient's performance at a glance, alleviating the clinician's need to compute scores into percentages and creating more objective data for growth comparisons.

Because the system utilizes smart devices typically used and carried by the patient, it can support activities across diverse geographical regions, and in particular at the patient's home and place of work. The system can also utilize a central server system programmed to provide detailed and innovative analysis of the data collected from the patient smart device obtained during patient exercises.

An important part of the innovative process is the use of artificial intelligence systems that learn from prior activities and collected data, and that provide smart and intelligent analytics from the collected data.

Machine learning can be utilized to assess and treat facial droop, for example. The system can leverage two specialized datasets to power an Artificial intelligence engine for decision making. These datasets will enable the system to detect facial droop in 3D scans as well as recommending exercises tailored to the patient's specific needs.

A dataset will be built from a collection of 3D facial Scan Data obtained from the patient and analyzed using custom software, such as OpenCV algorithms, and paired with DLIB (a modern C++ toolkit containing machine learning algorithms and tools used for creating the complex software analysis algorithms in C++ to provide the disclosed features). Each scan includes data on key facial features that are often affected by droop, such as: (1) Cheek to eye symmetry; (2) Lip asymmetry and/or (3) Jawline positioning, among others.

The culmination of a 3D scan data, which has been determined to have no facial droop, will be averaged out to make a baseline face. Other facial scan data with varying levels of droop will be categorized and checked against the new baseline 3D face.

The second dataset will be generated based on actual facial droop data from the patient, by severity and location, and effectiveness of certain exercises for these affected areas.

For example, an algorithm to analyze the data is configured to use Convolutional Neural Networks, trained via TensorFlow & PyTorch, to process the 3D mesh data and identify variations in the facial structure that indicate droop. The more scans that are processed, the better the system detects facial asymmetry and droop. The system will also utilize such data to analyze the current 3D facial scan so that it can correlate the results through the trained dataset to suggest the strongest exercises for the level of facial droop in the affected areas. For example, if the algorithm detects that cheek movement is weak, the system will suggest exercises targeting the cheek muscles. This can be done for any muscle or muscle group being monitored.

An innovative feature of the invention is the Face Mask. The Face Mask tool revolutionizes the treatment of facial droop by replacing subjective estimations used in conventional treatment with a quantitative, data-driven approach. Utilizing, for example, Apple's ARKit's 10,000-point facial mesh and machine learning, the system provides precise detection and tracking of facial asymmetry over time, establishing a reliable baseline profile for comparison. Clinicians benefit from objective, consistent data, reducing human error and enabling more accurate assessments. The tool's AI-powered recommendations offer targeted exercises, specific facial muscles, continuously adapting based on patient progress. As a result, the system generate assessment data can be used to generate an assessment report, including any of the results described herein for providing to the therapist, based on analysis of the patient exercise data to assess the patient progress, so that the patient receives more effective treatment plans leading to better recovery outcomes.

The exercises and other data collection routines collect the necessary data during patient exercises and activities to generate a point cloud of 3d mesh data. The 3d positions of vertices is recorded as the face takes different poses. Those are all stored alongside some metadata to point out date, time and pose. This can be provided as an encrypted text file with a desired file format that can be transmitted to the central server for analysis. Each time a patient uses the face mask tool this file is updated. The file is re-submitted to the server and the server immediately makes its assessment based on analyzing the updated data to assess the patient progress.

The smart device, executing the installed application, can access, decrypt and display the same mesh data as well as show the progression in different poses and timestamps. Both the patient and the therapist/doctor can interact with this data using the application running on a smart device, tracking the progress of the patient during treatment.

The central server can utilize various software tools to analyze the data collected by the patient's smart device, which can be sent to the central server via the Internet or any communication network (such as cellular services, radio, satellite, Wi-Fi, hardwire connections, or any means of connecting to the Internet, for example). The analysis tools can include, for example, the following:
TensorFlow: One of the primary machine learning frameworks used for training Convolutional Neural Networks (CNNs), which analyze 3D facial scans and identify facial droop patterns.
PyTorch: Another machine learning framework used alongside TensorFlow to enhance model training and ensure flexibility in developing the droop detection and exercise recommendation algorithms.
OpenCV: A computer vision library used to analyze facial scans, particularly for key facial features like cheek-to-eye symmetry, lip asymmetry, and jawline positioning.
DLIB: A tool paired with OpenCV for facial recognition and landmark detection, enabling accurate mapping of facial features and providing the necessary data for machine learning analysis.
Convolutional Neural Networks (CNNs): Used to analyze variations in the 3D mesh data, detect facial droop, and recommend exercises. The algorithm continuously improves as more data is processed, allowing for better detection and more personalized treatment recommendations.
Apple ARKit: ARKit's 10,000-point facial mesh is used to capture detailed 3D facial scans from the patient, such as during exercises, to capture patient exercise data. This provides a high-resolution model of the patient's face, allowing for accurate detection of facial droop and tracking changes over time.

Note that although the above state-of-the-art tools can be used for the example embodiments, additional tools and alternatives will become available for similar purposes, and will be utilized as available.

Appendix B of the '680 application, incorporated herein by reference, discusses various problems and improvements to the state-of-the-art provided by the disclosed system and method, as discussed above. One example system described in those incorporated materials is Speak2me Technology. This appendix describes reasons why the improved methodology works and how it improves the therapy process, including: Providing effective tasks with objective data collection to track progress and justify ongoing therapy needs to insurance companies; Boosting SLPs productivity standards; Providing billable teletherapy and progress tracking; Decreasing percentage of no-show due to transportation restraints; Improving face-mapping technology to better analyze the patient's progress; and Notifying changes in facial symmetry to alert the SLP if medical status has changed (i.e., stroke).

If the system detects problems in the patient during its recording and analysis activities, such as improper patient practices, deterioration (e.g. a further stroke), or other problems, it can immediately message the therapist and/or emergency personnel, as desired.

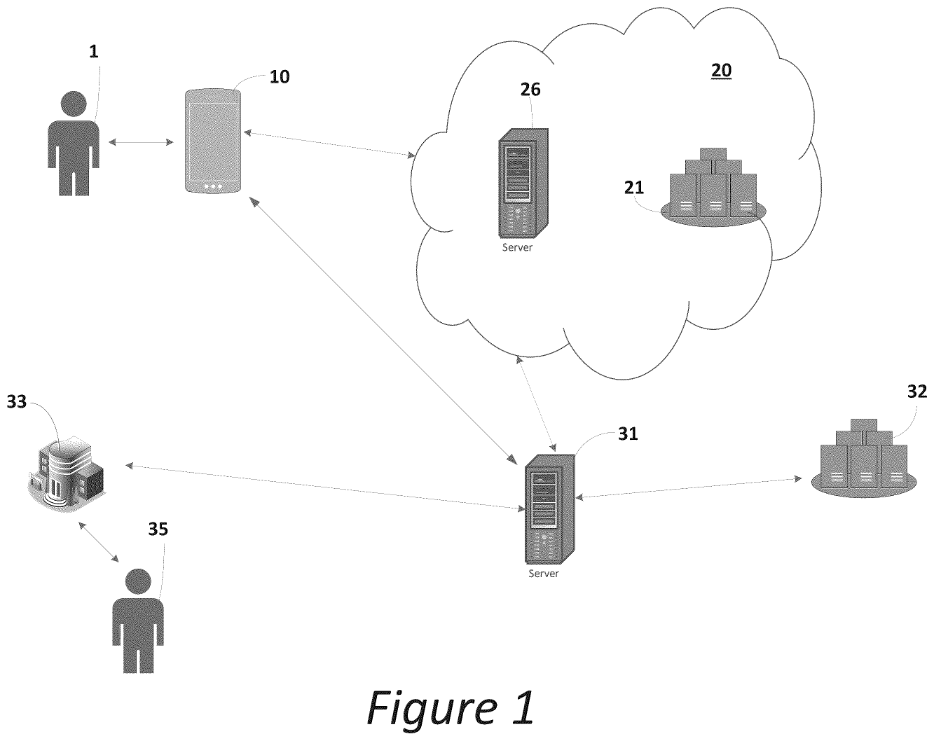

FIG. 1 shows an example system for implementing the improved methodology. The system can utilize one or more servers 31 using one or more databases 32 to provide a centralized system for performing an analysis of video and/or photographs taken of a patient 1 via a smart device (e.g., smartphone) 10 of the patient. By using the patient smart device 10, the patient can interact with the system by performing exercises and other activities that the therapy requires of the patient in any arbitrary location that the patient may desire, such as their home, office, care facility, park, or any other location the patient may take the smart device.

The system can utilize cloud-based services 20 such as various servers 26 and/or databases 21 to support the methodology. For example, services such as GOOGLE Play or Apple's App Store can be used for housing and installing one or more apps on the patient's smart device 10. Furthermore, development tools provided by various cloud services can be used as well. Note that the system server(s) 31 and database(s) 32 might also be part of various cloud-based services.

The system servers 31 can be programmed with specialized software to analyze the data collected from the patient smart device, including the use of artificial intelligence and learning algorithms, to generate assessment data from the patient data for use to provide assessments and assessment reports and tools for use by the medical provider 35 in monitoring and supporting the patient therapy. Such assessments can be automatically generated based on the collected patient data, with such automated assessments including the use of artificial intelligence tools and learning routines.

A medical provider 35, such as a therapist, doctor, nurse, medical aid, or other medical person can interact with the system using their own smart device(s) to execute software installed on those devices to monitor the patient progress collected by the system, including viewing face mask images and timeline videos created therefrom. The provider 35 can also provide updates to the system in response to the patient progress reports, such as changing or replacing the exercises for the patient to use.

Hence, the system operates by having a central server providing the primary data processing, with smart devices connecting to the central server (e.g., via a web portal) on both ends. The patient smart device is utilized to collect data from the patient via patient exercises and monitoring, while a medical provider smart device receives the analyzed data from the server for use by the medical provider to assess the patient's progress. The central server generates the images, reports, and other analysis data and tools for helping the therapist assess the patient progress. Note that the patient's device can also be utilized to show the patient the patient's progress as well, to provide motivation to the patient to continue the exercises and continue supporting the process.

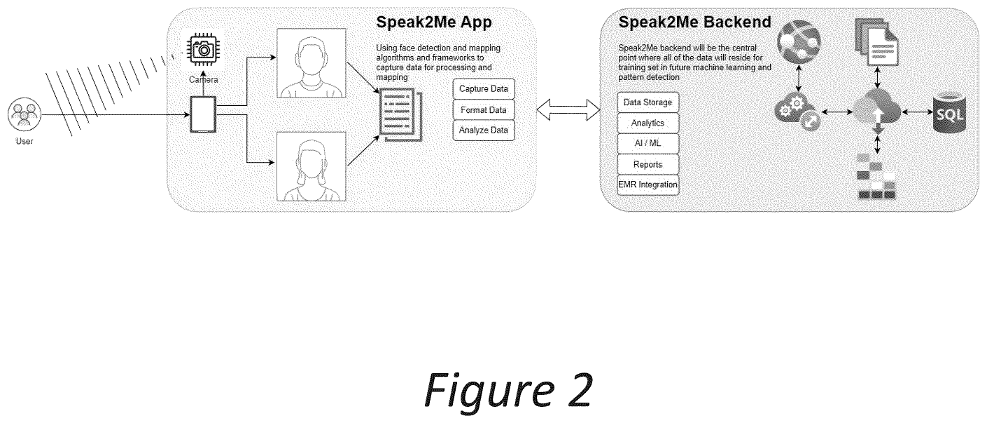

FIG. 2 provides a graphic showing an example of an application functionality that can be installed on the patient's smart device (and/or the medical providers smart device), along with backend functionality that can be centralized in a server, for example. Of course, any functions can be centralized other than the video and photograph applications that are likely provided as a part of the smart device. Alternatively, various functions that are shown in the central system can also be provided by an app on the patient's smart device. The actual distribution of functions will be chosen based on costs and computing capability, along with privacy and other considerations.

The platform can have several modular applications: e.g., a patient facing tool, an SLP tool, and an enterprise facing tool.

The patient will have access to the technology through their mobile smart device, e.g., an Apple IOS or an Android device. The user's device should have a front-facing camera with depth or Lidar technology. This will allow for a more accurate data capture from the patient to generate patient exercise data from patient exercises for analysis. For non-mobile devices, the users will require a camera with similar capabilities such as Intel's RealSense Depth Camera. Data capture can include patient responses, including inputs to smart device interfaces such as text (e.g., typing) or voice responses, video capture of the patient performing exercises, device generated data such as GPS, location, time and date, and other device data.

Once the patient performs the onboarding process, they will have access to the Face HeatMap technology. They will be able to use the front facing camera of their device to allow the system to start capturing the data points for analysis and pattern recognition. This data will be passed to our platform and processed on the backend server using our cloud services and machine learning technology.

Once the system processes the data, it can provide a recommendation of a suite of exercises to the patient and the SLP for review and approval.

The SLP facing application will provide a more in depth view of the data captured through our algorithm and they will be able to view reconstructed 3D models of the patients facial features. They will also have access to more detailed reports of the data and provide training sets for future enhancement of the algorithm.

Various features of the inventive system are described hereinbelow:

Patients can develop speech problems and other facial problems due to various pathologies, such as stroke or other injury to the brain or nervous system and muscles.

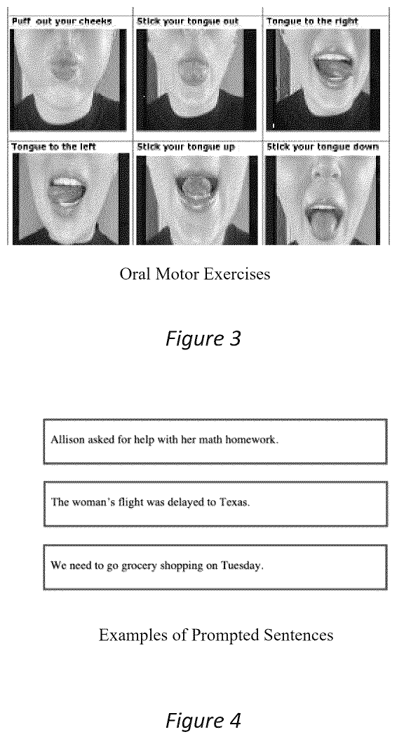

Once specific speech problems are identified, the therapist can begin by providing the patient with a series of oral motor exercises such as shown in FIG. 3, such as puffing out the cheeks, sticking out the tongue, moving the tongue to the right and/or the left, and up and/or down. These, and other, patient exercises are used with the patient smart device to generate and capture patient exercise data for further analysis to generate assessment data used to provide reports and tools for assessing the patient progress.

While engaging in the oral motor exercises, the patient's facial movement is to be closely observed by the therapist. The patient is monitored for any twitching, abnormal asymmetrical movements, drooping, and slowed movement, of either side of the face. Then, to exercise the facial muscles, the therapist prompts the patient to repeat words, phrases, and sentences, such as those shown in FIG. 4 as examples. The invention allows these exercises to be performed remotely from the medical provider and the analysis system using the patient's smart device, with the smart device monitoring and recording the patient's progress to capture and generate patient exercise data for analysis.

With the current practice, the typical tool available to the Speech-Language Pathologists (SLP) to view facial movement during speech is the naked eye, typically done at the therapist office or in the patient's home with the therapist present. This results in very limited metrics being available to the therapist, due to travel constraints, the lack of effective tools, and other problems. The therapist, prior to each session of speech therapy, will determine which parts of the face they deem as having improper movement. To accelerate improvement of the deemed improperly moving section in uniform with the whole face, using specific words that result in certain facial movements is key.

A current problem for the therapist using conventional techniques is that they are unable to track specific details of a patient's face by mere visual observation in their offices. As a patient speaks and repeats the prompted words, sentences, and phrases, the therapist is only able to observe one section of the patient's face at a time. This becomes challenging when the therapist is trying to initiate multiple parts of a patients face to move in unison with the rest of the face.

In addition, the patient's face often moves too fast for the therapist to be able to accurately gauge which specific sections of the face are moving at certain speeds. For example, a patient repeating the word “Apple” results in a different combination of facial movements in comparison to the word “Banana”. SLP's often have a library of these words, sentences, and phrases, which from experience, they know will trigger specific facial movements.

To trigger the desired facial movements, the therapist may, for example, prompt patient to say 3 words in order, such as (1) Apple; (2) Banana; and (3) Carrot. The patient verbalizes the three words, with the therapist observing resulting facial movements, such as: observing a droop on the patients left side of the face, and asking the Patient to repeat the word (e.g., “apple”) multiple times, seeing that when the word (“apple”) is spoken, the droop in the left side of the face appears to conform back to a somewhat normal position, etc. The therapist then prompts the patient to use the word in a sentence, such as saying “I would like to eat an apple.” The therapist can continue such exercises to see how the patient is progressing and what the problems that still exist may be.

In traditional practice, the therapist is taking notes and otherwise manually capturing his/her observations and assessments. These must manually entered into the medical database for housing medical records used by the therapist. This is time consuming and prone to data entry errors. In contrast, using the inventive process, the data is generated in digital format that can be easily (and automatically) converted into a form for direct entry into the medical database. This increases the efficiency of the overall process and improves the accuracy by reducing the potential for errors.

Hence, to improve the efficiency and effectiveness of the therapy, the invention involves automating many of the processes, allowing them to be performed remotely (e.g., at the patient's home and/or office or at a care facility) and collecting additional data, utilizing modern computing technologies. This data can be collected wherever the patient may be, using a mobile smart device of the patient with appropriate functions installed and/or enabled and executing specialized software provided by the system (when needed). Central servers and data storage, along with the patient's mobile smart devices running the specialized software, photography functions, and video and audio functions, can be utilized to automate and improve many of the necessary functions of speech therapy to benefit both the patient and the therapist. The therapist, also using a smart device, then accesses the data collected from the patient (e.g., via a central server), to monitor and assess the patient's progress.

An example pseudocode for a session can be as follows:
Initiate Face Detection
Initiate Proprietary Algorithm for Face Heat Map
For the duration of the analysis, continue capturing data points from the patient's face:
   a. Generate the geometry/mesh for the captured data points for visualization
   b. Feed captured data points into Speak2Me proprietary algorithm for pattern detection
   c. Store data into database for future analysis and machine learning data set
Store suggestions of therapy to be reviewed by SLP
Generate 3D models for further review and analysis

Using the improved process and smart devices, the therapist can create interactive exercises, such as via an iOS mobile application (Operating System for Apple iPhone and iPad), or Android on other smart devices, for the patient to practice, remotely or in person, in a manner similar to that of an interactive mobile game, while capturing information about the patient for use by the therapist, who need not be present during this process. The system, using artificial intelligence applications and other analysis tools, can help the therapist determine the progress of the patient, allowing the therapist to provide additional instructions, updated exercises and tools, new exercises and tools, and other therapies to improve the patient's progress. Appendix C of the '680 application, incorporated by reference, shows an example system utilizing the tools of Apple's ARKit, which is discussed in some detail hereinbelow.

Appendix D of the '680 application, also incorporated by reference, shows some additional example exercises.

For example, video and audio captured by the remote devices can be utilized to monitor the progress of the patient. When the patient manually presses a button that asks for consent to record, both the camera and microphone inputs of their smart device (e.g., iPhone or iPad or Android smartphone), the exercises are then displayed to the patient, and captured by the device for storage and later observation by the therapist. Such storage may be performed locally, and/or this data can be forwarded to a remote central server of the system for storage and/or analysis (such as via the Internet or the Cellular network, for example).

As a patient completes verbalizing various words, phrases, and sentences prompted by the device, such as those that before would be spoken to a therapist in person, these responses and the associated facial expressions and movements are recorded by the smart device, such as by an iPhone or an iPad Lidar Face Camera or an Android smart phone or tablet device, and the results stored and ultimately forwarded to the central server for analysis.

This recording of the practice session and resulting analysis (whether stored centrally, or on the patient's device, depending on the approach) can then be transmitted to the therapist, such as via the remote internet or cellular communication, to allow the therapist, using his own smart device executing a specialized software program, to view images and graphics of the face of the patient during the practice, including specific degrees of movement when certain parts of the face that are in motion, using, for example, the ARKit FaceGeometry, an open source tool provided by Apple (see referenced Appendix C of the '680 application). Other similar tools could be used for Android devices.

For example, a software framework can include:
Deploying and supporting both iOS and Android devices using the Flutter Framework that enables the deployment of single source code for targeting multiple devices and platforms.
Using Azure Cloud Services for all backend related deployments and services that will be implemented.
Using the built-in face recognition and detection framework for each platform for capturing the basic data points for processing.
Using ComputerVision technologies and frameworks to enhance the data capture and analysis prior to feeding them into our proprietary algorithm.

Utilizing Apple ARKit via the iPhone Lidar Camera, the iPhone/iPad is configured, for example, to track and record 1057 vertices and 1752 triangles overlaid on the users face. Using the facial model, the patients'face is overlaid with the ARFaceGeometry mesh as the patient completes the prompted exercises. In real time, 52 values pertaining to facial movement are recorded every 0.1 second. The tool iOS blendShapes dictionary provided by the ARFaceAnchor objects describe the facial expression of a detected face in terms of the movements of specific facial features (eyes, eyebrows, lips, chin).

For each key in the dictionary, the corresponding value is a floating point number indicating the current position of that feature relative to its neutral configuration, ranging from 0.000 (neutral) to 1.000 (maximum movement).

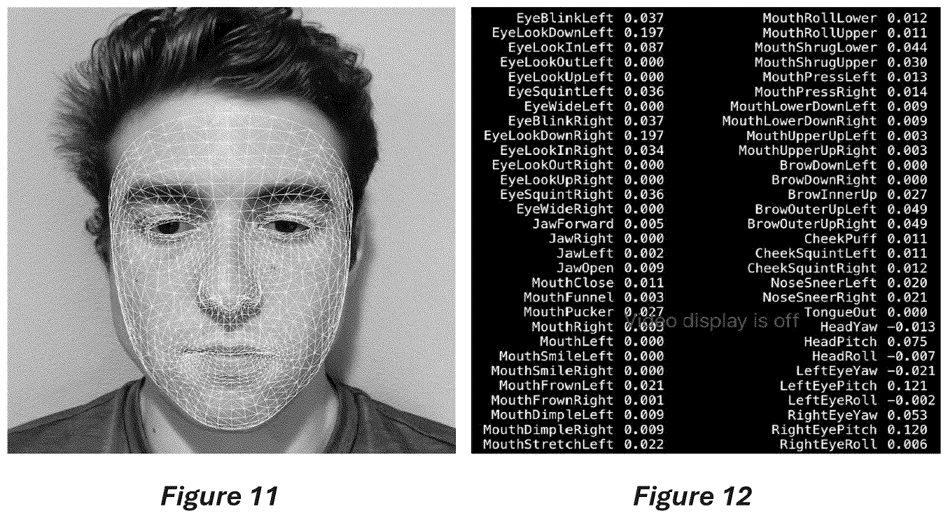

FIG. 11 shows an example face mesh with the monitored data points and their movement shown in FIG. 12. There are, for example, 26 total facial movements that are given individual points representing the minimum and maximum movement, on each side of the face, resulting in 52 values. Each blendShape is able to provide a complete mold that tracks essentially every possible movement to a degree of 0.1 mm or less.

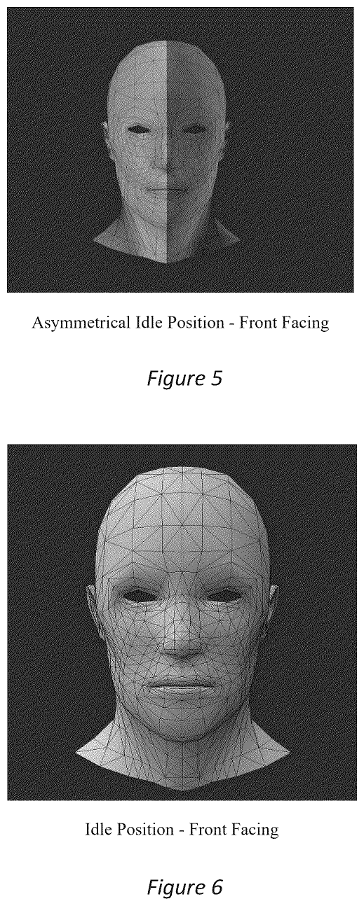

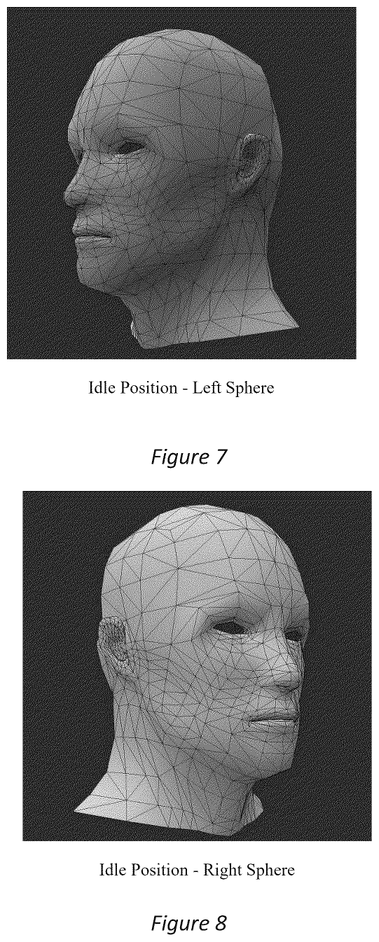

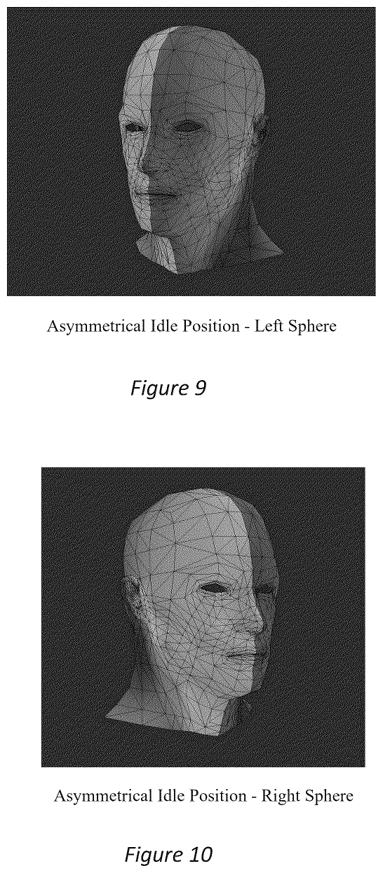

Facial movements located around the regions of the mouth, nose, and chin, are the visual target for the SLP. The ARKit is equipped with the following movements located around the mouth region, and are triggered as “in movement” during speech, for example (as shown in the examples of FIGS. 5-10):

For LEFT MOUTH, movements including mouthDimpleLeft; mouthFrownLeft; mouthLowerDownLeft; mouthPressLeft; mouthStretchLeft; mouthUpperUpLeft; can be recorded and viewed by the therapist.

Similarly, for RIGHT MOUTH: mouthDimpleRight; mouthFrownRight; mouthLowerDownRight; mouthPressRight; mouthStretchRight; mouthUpperUpRight; mouthRollUpper; mouthRollLower; mouthShrugLower; and mouthShrugUpper.

An exercise outline can be provided using ARKit Recording Patient Face to provide the therapist with information and visuals to determine patient progress. For example, the device can be used to prompt the Patient to say 3 words in order, as they appear on iPhone/iPad, such as: (1) Apple; (2) Banana; and (3) C: Carrot; while recording the patient's facial movements and expressions, and the speech sounds as the patient verbalizes “Apple” “Banana” and “Carrot” as prompted. As the patient completes verbalization, recorded using the iPhone/iPad front facing camera, the ARKit ARSCNFaceGeometry is overlaid on the patients face to generate an effective and accurate model.

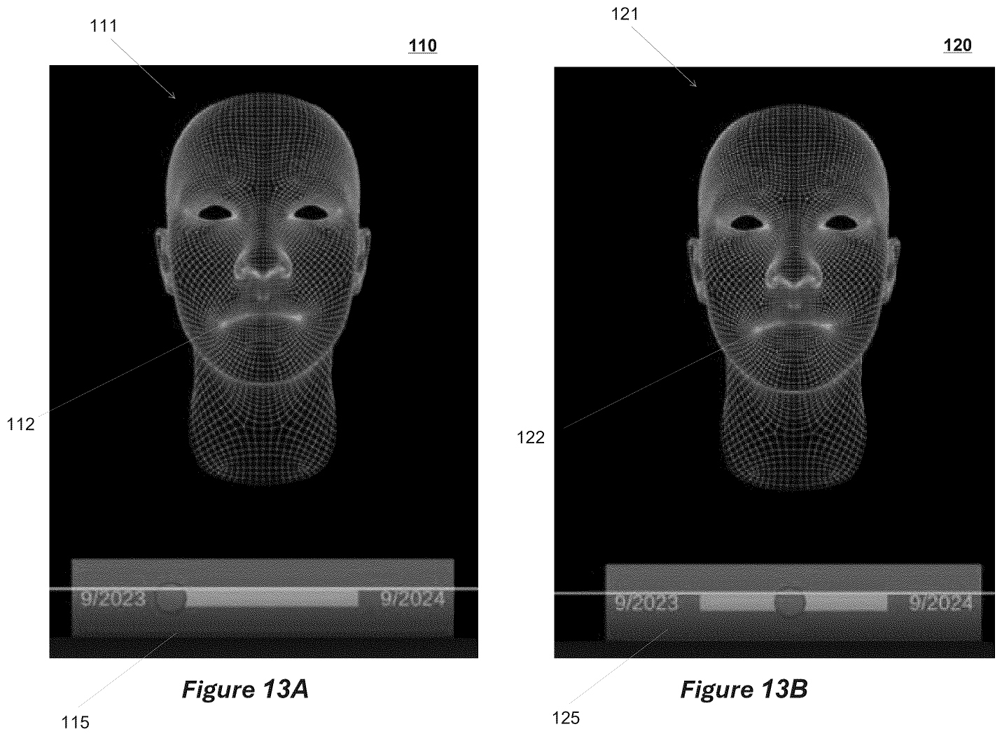

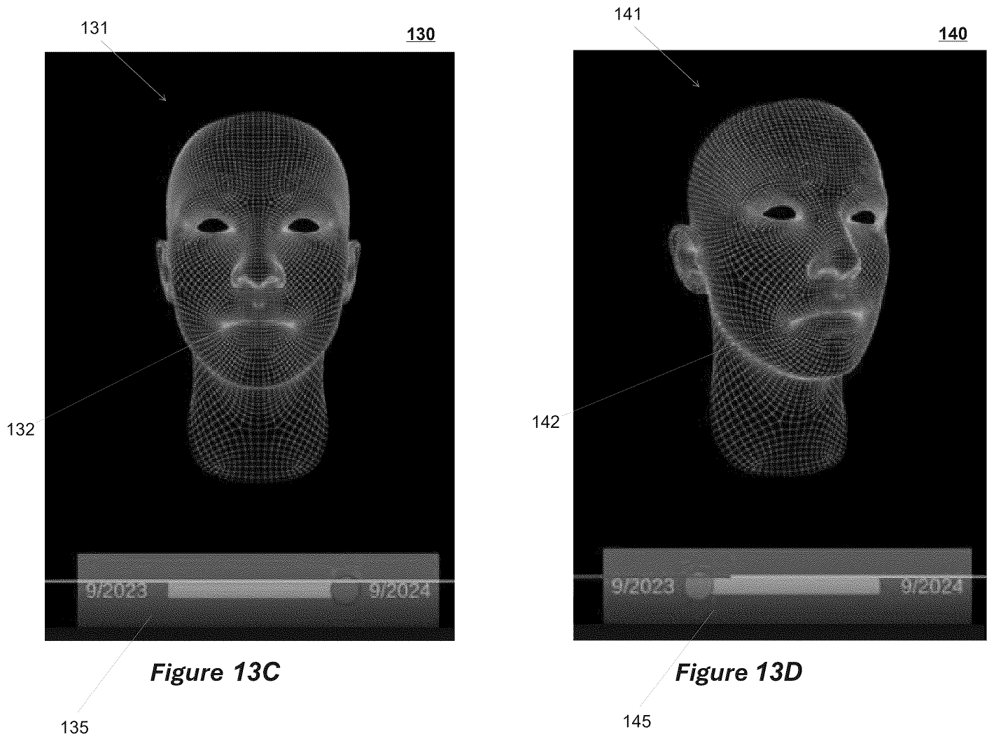

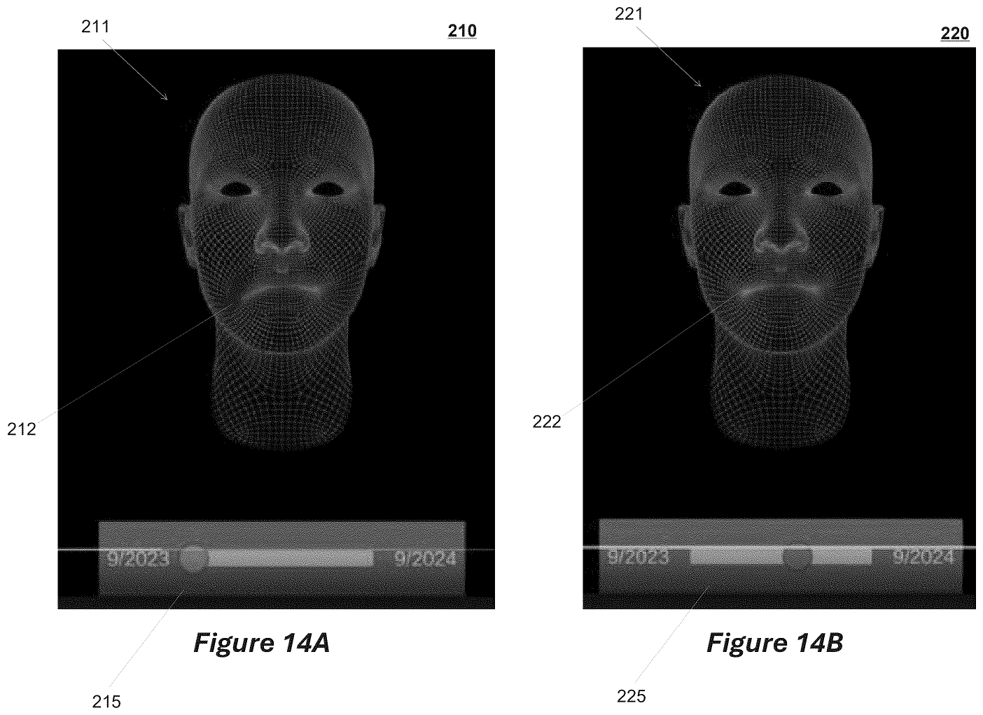

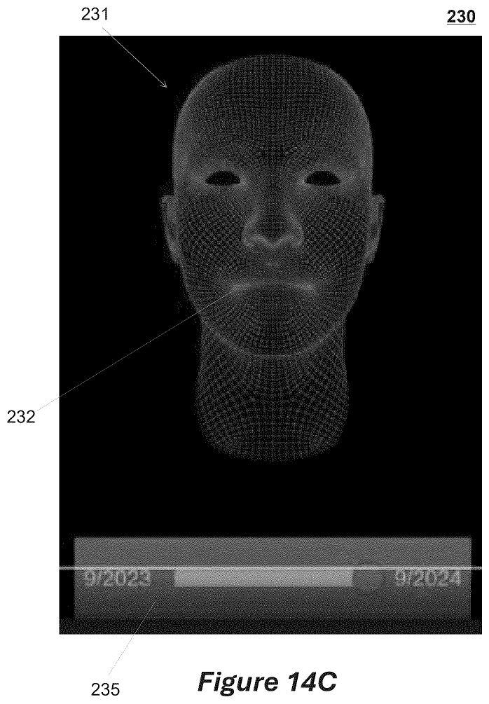

Using the recorded patient facial data, the system can generate patient assessment data that can include a time lapse video of the patient's progress over a period of time. FIG. 13A shows a screen capture 110 of facial mask image 111 taken on a particular date (in September of 2023). Note the droop in the mouth at 112. By moving the date slider 115 to the right, the image changes to the patient image capture 120 of the facial mask image 121 of FIG. 13B, with the slider moved forward in time, showing an improved droop 122. Finally, continuing to move the slider 135 as shown in FIG. 13C the patient image capture 130 shows mask 131 a year after the image 111 with an almost non-existent droop 132. Images can also be rotated to show other portions of the face, as shown in FIG. 14 with mask 141, showing mouth droop 142 in September 2023 set in slider 145. This is only one example of the many different mask images that can be generated for any area of interest in the face of the patient to show progress over time.

In practice, the ARSCNFaceGeometry assigns a value between 0.000 and 1.000 as the neutral base idle of the face as it is resting. 0.000 indicates minimum movement, and 1.000 indicates the total amount of possible movement.

As Patient says “BANANA” the ARSCNFaceGeometry records the values of each blendShape Word: BANANA

For example, these blendShapes are output to the screen when the ARSCNFaceGeometry mesh calculates the mesh vertices compressing and stretching in accordance with the following facial movements to the exact decimal degree of 0.005 blendShape Output: MOUTH DIMPLE LEFT 0.05; MOUTH DIMPLE RIGHT 0.05; MOUTH FROWN LEFT 0.05; MOUTH FROWN RIGHT 0.05; MOUTH DOWN LEFT 0.05; MOUTH DOWN RIGHT 0.05; MOUTH DOWN LEFT 0.05; MOUTH PRESS LEFT 0.2; MOUTH PRESS RIGHT 0.2; MOUTH STRETCH LEFT 0.2; and MOUTH STRETCH RIGHT 0.2.

Using 0.000 as a minimum baseline and 1.000 as a maximum ceiling, 26 facial movements (iOS blendShapes) are given accurate values that are updated every 0.1 seconds. The minimum and maximum values (0.000-1.000) are uploaded to a Unity via CSV file.

To support more detailed analysis, these values can be given corresponding colors, using a color scale to depict minimum and maximum values as a color scale “map” is overlaid on a users face, similar to that of a weather map. The 1057 vertices are fitted to a color coordinated system which allows for the minimum and maximum values to be visualized with a range of colors specific to each degree of movement.

The overlay of X/Y/Z coordinates on the users face assign values that translate to a scale of movement on a 3D model of the face, that is displayed via Unity, a 3D graphics and animation modeling software that is popularly used in video game development and animated video creation.
Using all 52 blendShapes, the 3D model is used to visualize each recorded movement in playback time. Warmer colors (Red/Orange/Yellow) are used to signify medium to maximum tension (0.500-1.000); Cooler colors (Purple/Blue/Green) are used to signify minimum to medium tension (0.000-0.500). RGB color values as such: 0.100-0.300 Purple; 0.3.00-0.500 Blue; 0.500-0.600 Green; 0.600-0.700 Yellow; 0.700-0.800 Orange; and 0.800-1.000 Red.
A split view of the face allows for SLP to detect for abnormal movement between the left and right spheres of the face, as well as to isolate certain blendShape movements
Different sensitivity settings enable coordinate tracking between certain facial features: —Eyes|Mouth; Mouth|Cheek; and Eyebrows|Jaw.

FIGS. 14A to 14C show example screen shots from an example color map time video that highlights the regions showing droop and muscle problems. FIG. 13A shows a screen capture 210 of facial mask image 211 taken on a particular date (in September of 2023) with color processing of the mask images. Note the droop in the mouth at 212 is highlighted with red pixels to show where the muscles are drooping (red because the droop is of a relatively high tension), with more red color where there is more droop. By moving the date slider 215 to the right, the image changes to the facial mask image 221 of FIG. 14B, with the slider moved forward in time, is a mask capture image 220 showing an improved droop 222 with less coloring moving into the purple range highlighting the reduced, but still present, drooping (purple shows less tension). Finally, continuing to move the slider 235 as shown in FIG. 14C shows the screen capture 230 of a patient mask 231 a year after the image 211 with an almost non-existent droop 232 shown by an entire blue image showing minimal tension. Images can also be rotated to show other portions of the face, as desired.

Hence, the system not only records, analyzes, and provides video and audio recordings of the patient that can be used for the therapist to review and analyze, but the system also uses these materials to generate patient assessment data used generate assessment data that includes raw data and also includes tools and automated analysis functions generated from the raw data that aid the therapist in evaluating and performing the therapeutic treatments.

In particular, the system can generate assessment data including a face mask for review by the patient and medical providers showing the operation of the patient's muscles and other facial features as determined during the exercises. Such a mask can be a time lapse video that interpolates progress continuously over time, and may include color codings for highlighting important features such as muscle droops. Examples of screen shots from an example face mask time video is shown in FIGS. 13A to 13C, and 14A to 14C, discussed elsewhere hereinabove.

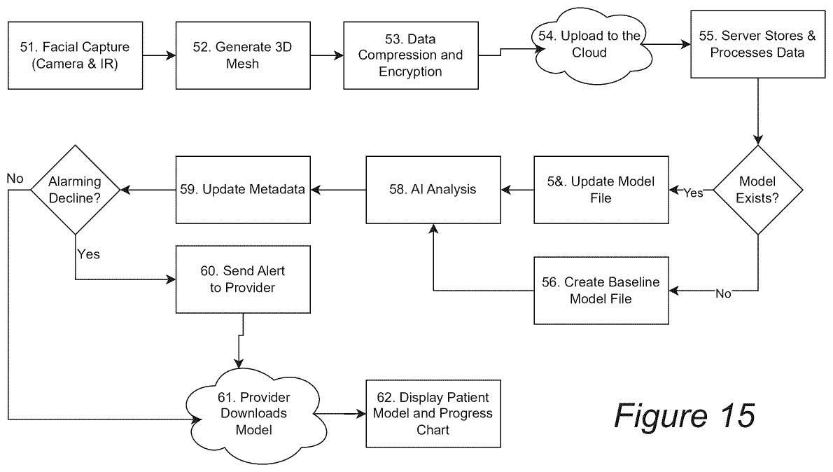

FIG. 15 shows a flow chart for one embodiment of the inventive process of supporting a speech therapy process, the example steps for which are described hereinbelow:

A Facial Capture (Camera & IR) step 51 utilizes the patient's device using both the camera and infrared (IR) sensor to capture a detailed 3D representation of the patient's face. The camera provides 2D visual data, while the IR sensor measures depth to generate an accurate 3D mesh of the face. This data forms the foundation for subsequent analysis of facial motion and any potential issues like facial droop.

A Generate 3D Mesh step 52 is used generate a 3D facial mesh from the collected facial data using specialized software executing on the patient smart device. Once the facial data is captured, the method can be used to combine the 2D and depth data to create a 3D mesh of the patient's face. This mesh maps thousands of points on the face, representing critical areas such as the eyes, mouth, jaw, and cheeks. This serves as a baseline for analyzing facial symmetry and movements.

A Data Compression and Encryption step 53 is used on the patient smart device to compress the 3D mesh and related data to reduce file size for efficient storage and transmission from the patient smart device to the central server system for further processing. The data is encrypted to ensure patient privacy and security during transmission, aligning with HIPAA compliance. This step ensures that sensitive information is protected before it is uploaded to the central server.

An Upload to the Cloud step 54 is used for uploading compressed and encrypted facial data to the “cloud” (e.g., on the central server) where it will be securely stored and processed. The cloud infrastructure provides accessibility for both the analysis process and remote access by healthcare providers.

A Server Stores & Processes Data step 55 provides that when data is uploaded, the server stores the data and begins processing it. This involves organizing the data into the patient's profile and preparing it for AI analysis. The server checks if a baseline model already exists, and either updates the existing model accordingly or creates a new one if necessary.

Create Baseline Model File step 56 operates if no previous data exists for the patient, such that the server generates a baseline model from the initial 3D mesh. This baseline serves as the reference point for all future scans, allowing the system to compare changes in facial structure and movement over time. Any future scans will be compared to this model to track progress.

The Update Model File step 57 operates If a baseline already exists, in which case the server updates the model with the new scan data. This updated model captures any changes in facial motion and structure since the last scan, enabling precise tracking of patient progress over time.

The AI Analysis step 58 provides smart processing of the collected data. The AI engine analyzes the updated 3D model by comparing the current facial structure against the baseline. The AI detects changes in facial symmetry, muscle movement, and droop severity. The AI uses this data to assess whether the patient's condition is improving, worsening, or remaining stable. The AI may also provide predictive insights based on previous cases using relevant data collected from other patients, for example.

The Update Metadata step 59 is for the system updating the metadata associated with the patient's file. This process can include information such as the date and time of the scan, analysis results, severity ratings, and any new insights. This metadata helps medical providers track patient progress and compare current results with past data points.

A Send Alert to Provider step 60 is used If the AI detects an alarming decline in the patient's condition or an unexpected change in the patient's progress. It might also be used if the patient indicates an urgent matter using his smart device, such as during an exercise. It provides an alert that is automatically sent to the provider for urgent treatment. This alert can provide the analysis results and flags any critical issues that may need immediate attention, and may provide suggested actions. The alert can be sent to the medical provider in various ways, such as by automated phone calling, texting, email, alerts to the medical provider smart device, or another method, any or all of which might be utilized to ensure that the medical provider is properly notified. Which of the patient's various medical providers need to be notified might also be determined and acted upon using the AI process.

A Provider Downloads Model step 61 notifies the healthcare provider when the updated model is available. The provider can securely download the model along with the analysis and metadata to the medical provider smart device(s), enabling them to review the patient's current condition, assess any changes, and make informed decisions about the next steps in treatment.

A Display Patient Model and Progress Chart step 62 is used to display information to the medical provider. The provider's device displays the updated 3D model along with a progress chart that shows how the patient's condition has changed over time. Visual tools such as heat maps may be used to highlight areas of improvement or concern, providing a clear and concise way for the provider to review the data and adjust the therapy plan accordingly. Suggestions for new or modified exercises or treatments might be provided to the medical provider.

These steps are merely one example of the methodology for providing the features and benefits discussed herein. Other steps, additional steps, or modified steps could also be utilized, as desired, to obtain the disclosed features and benefits.

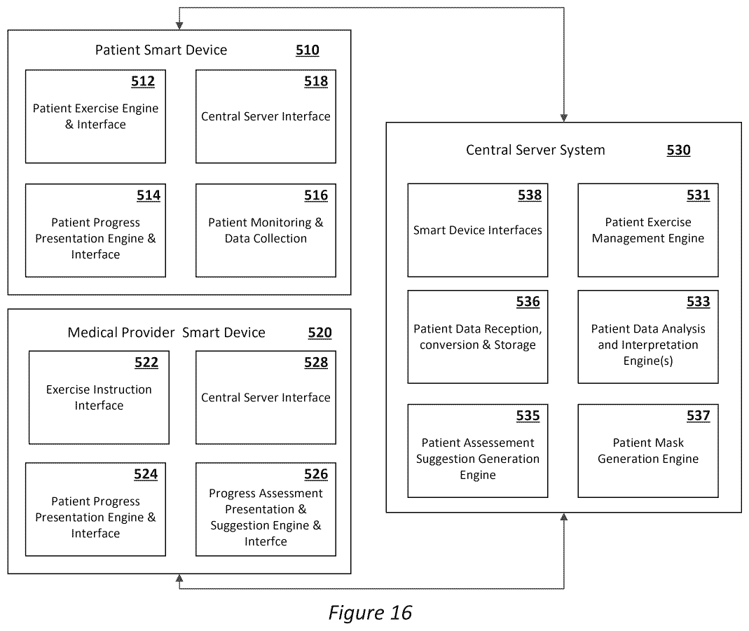

FIG. 16 is a block diagram showing one example architecture of an example embodiment for practicing supporting a speech therapy process. For the example embodiment 500, one can utilize a central server system 530 connected to both a patient smart device 510 (e.g., a smartphone, tablet, computer, etc.) and a medical provider (e.g., doctor or therapist) smart device 520 (e.g., a smartphone, tablet, computer, etc.). These are connected using one or more communications networks (e.g., cellular network(s), Internet, or any other appropriate communication network). These devices will execute software modules such as those described hereinbelow to perform their functions as described for the patient and the medical provider in this document and the incorporated materials. In particular, this architecture can be used to implement the process described by the flow chart of FIG. 15, for example.

The specialized software for performing these features on the smart devices can be provided and obtained at traditional software application providers, like, for example, Google's Play store or Apple's App store, where the patient and/or medical provider can download the application(s) for installation on their respective device(s).

Each device 510, 520, 530 will be connected to the respective communication network(s) using a respective communication interface 518, 528, 538. The interfaces connect the patient's smart device and the medical provider's smart device to the central server, preferably using secure and encrypted networks, for sharing data and information to support the therapy process, as discussed herein. These interfaces can be developed using the appropriate tools, such as using Unity for cross-platform mobile app development, supporting both iOS and Android. The communication between devices and the server can rely on RESTful APIs developed in Python (Flask/Django). Data encryption can use TLS/SSL protocols for secure transmission. Each interface will utilize the appropriate hardware to connect to its respective communication network. As pointed out, such networks can include cellular networks, Internet/Intranet networks, or any other appropriate communication network (e.g., satellite, radio, etc. using any appropriate communication protocol).

The Patient Smart Device 510 can utilize a Patient Exercise Engine & Interface module 512 which provides the exercises to the patient for viewing/practicing by displaying video and audio and graphics and text, as desired on the patient's smart device 510 using appropriate interface tools. This device may use features and components of the smart device such as displays, speakers, GPS, time stamps, cameras, microphones, keyboards, etc. for interacting with the patient. This engine can be implemented, for example, in Unity for interactive game-like exercises. For machine learning, TensorFlow Lite can be used to ensure AI processing is efficient on mobile devices. Exercises will pull data from the central server and feed back user results to that central server for further processing.

The Patient Smart Device 510 can also utilize a Patient Progress Presentation Engine & Interface module 514 is used to display patient progress, including performance metrics and video content received from the central server 530, through, for example, a React/React Native interface. Data visualization libraries like D3.js can be used for dynamic charts, while AWS S3 can be used to handle video storage and streaming. Hence, this module helps the patient monitor his or her progress using graphics, video, audio, etc., in a manner that can be similar or identical to that provided to the medical provider, as discussed in this document.

The Patient Smart Device 510 can also utilize a Patient Monitoring & Data Collection module 516 for recording patient data during exercises (as discussed herein) using the smart device 510 interface tools through, for example, Unity for game interactions and using ARKit (or ARCore for Android) for face-tracking inputs. The data can be processed and encrypted using AES-256 encryption before being sent to the central server 530 via an API Gateway on AWS. This data is used by the central server 530 for further processing and analysis as discussed herein.

The Medical Provider Smart Device 520 can utilize an Exercise Instruction Interface module 522 which lets the Medical Provider select and assign exercises for the patient, such as through a React front-end with a Node. js or Python back-end. The exercises can be stored in PostgreSQL databases, with GraphQL for querying exercise data on the central server system 530, for example.

The Medical Provider Smart Device 520 can utilize a Patient Progress Presentation Engine & Interface module 524 which can be used to display the patient progress information and analyses conducted by he Central Server System 530 based on the data collected by the patients, to both patients (on their respective smart devices) and providers, as discussed herein. It can be implemented using React and leverages AWS Lambda for processing data in real-time. It uses WebSockets for real-time updates and D3.js for data visualization.

The Medical Provider Smart Device 520 can utilize a Progress Assessment Presentation & Suggestion Engine and Interface module 526, which provides the Medical Provider with videos, suggestions, and assessments, using patient data processed on the Central Server System 530 through AWS, for example, to power machine learning models for providing such suggestions and assessments. These models analyze patient progress and generate suggestions based on historical data (which can include data about the particular patient and/or other patients) and current data (which can include data about the particular patient and, if desired, other patients as well).

Most of the data processing will be conducted by The Central Server System 530 which will utilized commercial databases, custom analysis tools, and artificial intelligence tools for performing smart analysis of the therapy process and patient progress. This server 530 can utilize a Patient Exercise Management Engine 531 manages and tracks exercises assigned to the patient. Built using Python or Node. js for backend logic, PostgreSQL can be used to store the patient's exercise history, and Celery will manage task queues and reminders for exercises.

The server 530 can utilize a Patient Data Reception, Conversion, and Storage module 536 which manages patient data, encrypts it, and stores it in a central database(s). AWS RDS (PostgreSQL) can be used for structured data, and AWS S3 for file storage (e.g., videos). AWS KMS ensures data is encrypted both in transit and at rest.

The server 530 can utilize a Patient Data Analysis and Interpretation Engines module 533 to analyze the patient data, including voice and facial movements collected by the patient smart device. AI models built using TensorFlow and PyTorch can be used to interpret the data and provide insights on progress. These models can be trained using data collected over time about both a particular patient and other patients to become more accurate as more data is gathered.

The server 530 can utilize a Patient Assessment Suggestion Generation Engine module 535 which takes the analysis results and converts them into reports and suggestions for the Medical Provider, such as noting patient progress (or lack thereof), suggesting new exercises, or modifying existing ones, for example (as discussed herein). Module 535 can use AI-based decision-making models trained via AWS SageMaker, for example, for providing suggestions to the Medical Provider via the Medical Provider smart device 520 through the secure interface.

The server 530 can utilize a Patient Mask Generation Engine module 537 which generates the patient mask and heat maps for monitoring facial symmetry and recovery progress (as discussed herein). ARKit (iOS) or ARCore (Android) can be used to collect facial data from the patient smart device 510, while the 3D mask can be generated in Unity. The data is processed and visualized, for example, using OpenCV for image processing and analysis.

Note that FIG. 16 provides just one example embodiment for implementing the inventive features described herein, but that other embodiments can be utilized as well, depending on the availability of hardware and software tools and services, which may be changed and even improved in the future.

This system can also be utilized to generate SOAP reports. In clinical speech therapy, documenting patient interactions using SOAP notes (Subjective, Objective, Assessment, Plan) is an important part of the process. Currently, SLPs spend a significant amount of time manually creating these notes after each session, and for many, the documentation process is an administrative burden. SOAP notes, while relatively quick to write, can add up to a time-consuming process, especially when managing multiple patients. Progress notes, which summarize patient data over time for compliance and billing purposes, take even longer to generate and present a greater challenge for therapists working in busy environments.

This system central server can provide automated SOAP note generation which offers an innovative way to simplify the documentation process for SLPs. By leveraging machine learning and AI, the system can collect, analyze, and synthesize patient data into a SOAP format with minimal manual input. It utilizes comprehensive analytics data recorded during each exercise session to provide an accurate and personalized report for SLPs. Unlike static documentation, the system dynamically adjusts based on the specific data points gathered from each exercise, ensuring a detailed and customized report for every patient interaction. This feature not only saves time but also improves the accuracy and consistency of patient documentation, reducing the risk of human error.

The SOAP note structure breaks down into four key sections: (1) Subjective (S): The patient's reported experience and feedback during the session; (2) Objective (O): Quantitative data collected during therapy, including performance metrics and exercise results; (3) Assessment (A): The clinician's analysis of the patient's progress based on subjective and objective data; and (4) Plan (P): The next steps in treatment, including adjustments to therapy and new goals.

The SOAP reporting is done through an automated process that includes: (1) Data Collection: During each exercise session, data is automatically gathered from patient exercises and interactions within the system platform. This data includes voice analysis, exercise results, and facial recognition data (if applicable); (2) Machine Learning Integration: The system uses an AI-driven algorithm to process the collected data and populate the Objective and Assessment sections. The AI also provides recommendations for the Plan based on patient progress; and (3) Pre-Filled Templates: Clinicians can quickly review and approve pre-filled SOAP notes, reducing the time needed for manual documentation.

Progress notes, typically required for compliance and billing every 90 days, summarize a patient's progress over time. These notes are critical for remote therapeutic monitoring (RTM) billing and are the most time-consuming documents for SLPs to generate. The system automates the creation of progress notes by compiling data from previous SOAP notes, tracking progress on goals, and analyzing overall patient performance.

Primary features of this SOAP process include: (1) Data Aggregation—The system pulls data from multiple SOAP notes, tracking goal completion and patient improvements; (2) Reminder System—Automatic reminders are sent to SLPs when progress notes are due, ensuring timely submissions for compliance and continued patient care; and (3) Customizable Timeframes—SLPs can set custom time frames for progress note generation, depending on patient needs or insurance requirements.

The system platform uses machine learning executing on the central server to automate SOAP and progress report generation. The algorithm is trained using anonymized data collected from real patient sessions, including good and bad examples of documentation, and incorporates facial recognition and voice analysis technologies for objective data collection.

A training dataset for use can include: (1) SOAP Note Data—Sample SOAP notes, both good and bad, are used to train the system on patterns in patient progress, assessment, and planning; (2) Exercise Results—Data collected from patient exercises is used to feed the AI, which learns to identify key performance indicators for each domain of speech therapy; and (3) Facial Recognition and Voice Data—Analyzes patient facial movements and voice patterns to further enhance the Objective and Assessment sections.

The technologies that can be used for providing the feature include: (1) Neural Networks—The system uses neural networks to analyze patient performance and automate SOAP and progress note creation; (2) Voice Analysis—Integrated voice analysis software, such as Whisper API, transcribes patient speech and provides real-time data on performance; (3) Facial Recognition—Apple's ARKit and 3D facial mesh are used to track facial movements and feed data into SOAP notes; and (4) Cloud Infrastructure—Data storage and processing are handled securely using AWS Cloud, ensuring compliance with HIPAA and other privacy regulations.

The automation of SOAP and progress reports significantly reduces the administrative burden on SLPs, allowing them to focus more on direct patient care. By collecting real-time performance data during therapy, Speak2Me ensures that notes are both accurate and timely, helping to improve the overall quality of care. The AI-driven approach minimizes errors and provides personalized treatment plans, making it a valuable tool for therapists seeking to improve outcomes for their patients. The system also increases efficiency, allowing clinicians to serve more patients while maintaining high-quality documentation.

The system can be monetized in various ways, including through individual subscriptions, SLP/Clinic subscriptions, and even an Enterprise subscription. Software applications might be sold or leased. In particular, compensation from medical insurance and/or Medicaid or Medicare is desirable, and the system can support remote monitoring CPT codes. Since the system provides financial, time, and travel efficiencies and improved patient care and results by monitoring patients in their own arbitrary locations where they conduct their exercises, reducing many of the otherwise traditional trips to the medical provider and/or otherwise increasing the ability to monitor patient progress without such visits, costs can be reduced while patient results are improved.

Although the invention has been described with particular focus on the field of speech therapy, it is noted that these techniques can also be applied to other forms of therapy and patient treatment.

For example, this process can be utilize to support the treatment of autism disorders by allowing a therapist or teacher to observe the autistic patient in various situations, including reacting to his/her environment, lessons and treatments, the effects of medications, and other activities. This allows the practitioner to observe the patient in a natural environment, and hence can provide a more accurate assessment of progress.

In addition, this process could be utilized for neurological treatments and psychological treatments in a similar manner. Patients can be remotely assessed for assessments of various exercises, treatments, medication impacts, effectiveness, and side effects, etc. Again, the patient can be observed in a natural environment to provide more accurate assessments than might be obtained in a clinic, for example.

Furthermore, this approach can be utilized for physical therapy as well. The patient can be monitored for physical exercises, medication impacts, etc. so that the progress of the patient can be monitored more regularly, and again in a more natural environment. In this manner, recovery from broken bones, joint replacements, tendon and ligament surgeries, muscle damage, etc. can all be monitored. Treatments for heart attacks and other major organ problems can also be remotely monitored for effectiveness and impact on the patient. In fact, any treatment provided to a patient that requires monitoring patient progress and regular treatments can utilize this technique to improve efficiency and accuracy and reduce the need for office visits.

As will be appreciated by one of skill in the art, the example embodiments may be actualized as, or may generally utilize, a method, system, computer program product, or a combination of the foregoing. Accordingly, any of the embodiments may take the form of an entirely hardware embodiment, an entirely software embodiment (including firmware, resident software, microcode, etc.) for execution on hardware, or an embodiment combining software and hardware aspects that may generally be referred to herein as a “system.” Furthermore, any of the embodiments may take the form of a computer program product on a computer-usable storage medium having computer-usable program code embodied in the medium. The various computer functions can be centralized, or distributed to mobile devices, as desired.

Any suitable computer usable (computer readable) medium may be utilized for storing the software. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet. Note that the computer usable or computer readable medium could even include another medium from which the program can be electronically captured, via, for instance, optical or magnetic scanning for example, then compiled, interpreted, or otherwise processed in a suitable manner, if necessary, and then stored in a computer memory of any acceptable type.

In the context of this document, a computer usable or computer readable medium may be any medium that can contain, store, communicate, propagate, or transport the program for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, radio frequency (RF) or other means.

Computer program code for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, Object Pascal, or the like, artificial intelligence languages such as Prolog, a real-time embedded language such as Ada, or even more direct or simplified programming using ladder logic, an Assembler language, or directly programming using an appropriate machine language.

Appropriate programming tools and languages and libraries and routines can be used to design the appropriate software for executing the desired functionality. Tools such as Unity for game interactions and ARKit for Apple (or ARCore for Android) for data inputs can be utilized. The data can be processed and encrypted using AES-256 encryption before being sent to the central server 530 via an API Gateway on AWS. RESTful APIs developed in Python (Flask/Django) and Data encryption using TLS/SSL for communication interfaces. AI models can be built using TensorFlow and PyTorch, for example. AI-based decision-making models can be trained via AWS SageMaker, for example. Display interfaces can be implemented using React and leverages AWS Lambda for processing data in real-time. WebSockets can be used for real-time updates and D3.js for data visualization. AWS can be used to power machine learning models for processing collected data. Python or Node. js for can be used for backend logic for various features and PostgreSQL can be used storing history, and Celery can be used to manage task queues and reminders for exercises. AWS RDS (PostgreSQL) can be used for structured data, and AWS S3 for file storage (e.g., videos). AWS KMS can be used to ensure that data is encrypted both in transit and at rest.

Examples of various embodiments may be described with reference to flowchart illustrations and/or block diagrams of methods, apparatus (systems) and computer program products according to the various embodiments. The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It will be understood that each block of the flowchart illustrations and/or block diagrams, and combinations of blocks in the flowchart illustrations and/or block diagrams, can be implemented by computer program instructions. These computer program instructions may be provided to a computing device including one or more processors of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems which perform the specified functions or acts, or combinations of special purpose hardware and computer instructions. The organization of such charts is for descriptive purposes for ease of communication, whereas the actual organization of the software code for some embodiments may utilize any of many different organizational structures that may differ substantially from the examples.

The computer program instructions may be stored or otherwise loaded in a computer-readable memory that can direct a computing device or system, or other programmable data processing apparatus, to function in a particular manner, such that the instructions stored in the computer readable memory produce an article of manufacture including instruction means which implement the function/act specified in the flowchart and/or block diagram block or blocks.

The software comprises computer program instructions that are executed by being provided to an executing device or component, which can include a processor of a general purpose computer, a special purpose computer or controller, or other programmable data processing apparatus or component, such that the instructions of the computer program, when executed, create means for implementing the functions/acts specified in the flowchart and/or block diagram block or blocks. Hence, the computer program instructions are used to cause a series of operations to be performed on the executing device or component, or other programmable apparatus to produce a computer implemented process such that the instructions which execute on the computer or other programmable apparatus the steps for implementing the functions/acts specified in this disclosure. These steps or acts may be combined with operator or human implemented steps or acts and steps or acts provided by other components or apparatuses in order to carry out any number of example embodiments of the invention

The terminology used herein is for the purpose of describing example embodiments only and is not intended to be limiting. As used herein, the singular forms “a”, “an” and “the” are intended to include the plural forms as well, unless the context clearly indicates otherwise. It will be further understood that the terms “comprises” and/or “comprising,” “including,” “having,” “containing,” when used in this specification, specify the presence of stated features, integers, steps, operations, elements, and/or components, but do not preclude the presence or addition of one or more other features, integers, steps, operations, elements, components, and/or groups thereof.

Many other example embodiments can be provided through various combinations of the above described features. Although the embodiments described hereinabove use specific examples and alternatives, it will be understood by those skilled in the art that various additional alternatives may be used and equivalents may be substituted for elements and/or steps described herein, without necessarily deviating from the intended scope of the application. Modifications may be necessary to adapt the embodiments to a particular situation or to particular needs without departing from the intended scope of the application. It is intended that the application not be limited to the particular example implementations and example embodiments described herein, but that the claims be given their broadest reasonable interpretation to cover all novel and non-obvious embodiments, literal or equivalent, disclosed or not, covered thereby.

What is claimed is:

1. A method of evaluating a speech therapy treatment for supporting the speech therapy of a patient, comprising the steps of:
executing software on a patient smart device for performing the steps of:
providing one or more speech exercises to the patient,
the patient performing said one or more speech exercises using the patient smart device at an arbitrary location of the patient,
said patient smart device monitoring the performance of the patient performing said one or more speech exercises, said monitoring including capturing responses of the patient while performing the exercises to generate patient exercise data, and
the smart device sending the patient exercise data over a communication network to a remote server;
said remote server receiving said patient exercise data;
said remote server executing software for performing the steps of:
analyzing said patient exercise data for determining patient progress,
generating assessment data of the patient progress based on said analyzing, and
the server sending the assessment data over a communication network to a medical provider smart device of a medical provider located remotely from the patient; and
said medical provider smart device providing said assessment data for access by the medical provider to review the progress of the patient regarding the speech therapy treatment.

2. The method of claim 1, wherein said assessment data includes data for generating a patient face mask of the face of the patient.

3. The method of claim 1, wherein said assessment data includes data identifying patient facial droop(s) shown by the exercises.

4. The method of claim 3, wherein said assessment data includes one or more images of the face of the patient showing an extent of patient facial droop(s) shown by the exercises.

5. The method of claim 3, wherein said assessment data includes one or more images of the face of the patient providing color indications identifying an extent of patient facial droop(s) shown by the exercises.

6. The method of claim 5, wherein said assessment data includes a plurality of said images of the face of the patient at different times.

7. The method of claim 6, wherein said plurality of said images is formed into a video timeline for viewing by the medical provider to show patient progress from the treatment over time.

8. The method of claim 1, wherein said assessment data includes an automatically generated assessment of the progress of the treatment for the medical provider.

9. The method of claim 1, wherein said assessment data includes an automatically generated assessment report of the progress of the patient.

10. The method of claim 9, wherein automatically generated assessment report includes suggested changes in said therapy determined using a machine based learning algorithm executing on said remote server.

11. The method of claim 1, wherein said speech exercises include oral motor exercises that are recorded by the patient smart device for generating said patient exercise data.

12. The method of claim 1, wherein said speech exercises include using said patient smart device for displaying images to said patient and requesting a patient response to said images for generating said patient exercise data.

13. The method of claim 1, wherein said speech exercises include using said patient smart device for detecting a speech impediment including one or more of stuttering, an inability to recite words; and/or forgetting words.

14. The method of claim 1, further comprising the step of the central server generating a SOAP report for the medical provider.

15. A method of evaluating a speech therapy treatment for supporting the speech therapy of a patient, comprising the steps of:
capturing data from images of the face of the patient for generating a patient baseline for the patient;
executing software on a patient smart device for performing the steps of:
providing one or more speech exercises to the patient,
the patient performing said one or more speech exercises using the patient smart device at an arbitrary location of the patient,
the patient smart device monitoring the performance of the patient performing said one or more speech exercises over time, said monitoring including capturing responses of the patient while performing the exercises to generate patient exercise data, and
the patient smart device sending the patient exercise data over a communication network to a remote server;
said remote server receiving said patient exercise data;
said remote server executing software for performing the steps of:
analyzing said patient exercise data for determining patient progress by comparing said patient exercise data taken over time with said patient baseline,
generating an assessment report of the patient progress based on said analyzing, wherein said assessment report includes an automatically generated assessment of the progress of the patient, and
the server sending the assessment report over a communication network to a medical provider smart device of a medical provider located remotely from the patient; wherein
said assessment report is provided to said medical provider to update the therapy treatment being provided to the patient.

16. The method of claim 15, wherein said update to the therapy treatment includes the steps of:
using said medical provider smart device to send updates to said speech exercises to said remote server over a communication network; and
said remote server sending one or more updates for speech exercises to said patient smart device over a communication network.

17. The method of claim 16, wherein said method is performed using the updates of the speech exercises to generate patient exercise data.

18. The method of claim 16, wherein automatically generated assessment includes suggested changes to said therapy determined using a machine based learning algorithm executing on said remote server, and wherein said updates for said speech exercises are determined utilizing said suggested changes.

19. The method of claim 15, wherein automatically generated assessment includes suggested changes in said therapy determined using a machine based learning algorithm executing on said remote server.

20. The method of claim 15, wherein said assessment report includes data for generating a patient face mask of the face of the patient.

21. The method of claim 15, wherein said assessment report includes data identifying patient facial droop(s) shown by the exercises.

22. The method of claim 21, wherein said assessment report includes one or more images of the face of the patient showing an extent of patient facial droop(s) shown by the exercises.

23. The method of claim 22, wherein said assessment report includes a plurality of said images of the face of the patient at different times.

24. The method of claim 23, wherein said plurality of said images is formed into a video timeline for viewing by the medical provider to show patient progress from the treatment over time.

25. The method of claim 21, wherein said assessment report includes one or more images of the face of the patient providing color indications identifying an extent of patient facial droop(s) shown by the exercises.

26. The method of claim 15, wherein said speech exercises include oral motor exercises that are recorded by the patient smart device for generating said patient exercise data.

27. The method of claim 15, wherein said speech exercises include using said patient smart device for displaying images to said patient and requesting a patient response to said images for generating said patient exercise data.

28. The method of claim 15, wherein said speech exercises include using said patient smart device for detecting a speech impediment including one or more of stuttering, an inability to recite words; and/or forgetting words.

29. The method of claim 15, further comprising the step of the central server generating a SOAP report for the medical provider.

30. A method of evaluating a speech therapy treatment for supporting the speech therapy of a patient, comprising the steps of:
executing software on a patient smart device for performing the steps of:
providing one or more speech exercises to the patient,
the patient performing said one or more speech exercises using the patient smart device at an arbitrary location of the patient,
said patient smart device monitoring the performance of the patient performing said one or more speech exercises over time, said monitoring including capturing responses of the patient while performing the exercises to generate patient exercise data, and
the smart device sending the patient exercise data over a communication network to a remote server;
said remote server receiving said patient exercise data;
said remote server executing software for performing the steps of:
analyzing said patient exercise data for determining patient progress by utilizing said patient exercise data taken over time,
generating an assessment report of the patient progress based on said analyzing, wherein said assessment report includes:
suggested changes to said therapy determined using a machine based learning algorithm executing on said remote server, and
data for generating a patient face mask of the face of the patient changing over time; and
the server sending the assessment report over a communication network to a medical provider smart device of a medical provider located remotely from the patient; wherein
said assessment report is provided to said medical provider to evaluate the therapy treatment being provided to the patient.
